(19) United States
(12) Patent Application Publication
(10) Pub. No.: US 2019/0302079 A1
(43) Pub. Date: Oct. 3, 2019

(54) MEASUREMENT APPARATUS

(71) Applicant: NGK SPARK PLUG CO., LTD., Nagoya-shi (JP)

(72) Inventor: Kaoru HISADA, Nagoya-shi (JP)

(73) Assignee: NGK SPARK PLUG CO., LTD., Nagoya-shi (JP)

(21) Appl. No.: 16/367,664

(22) Filed: Mar. 28, 2019

(30) Foreign Application Priority Data
Mar. 30, 2018 (JP) 2018-068295

(51) Int. Cl. G01N 33/00; G01M 15/10

(52) U.S. Cl. CPC G01N 33/0073; G01M 15/102; B60R 16/03; G01N 33/0054; G01N 33/0037

(57) ABSTRACT

A portable measurement apparatus which can be brought into a vehicle and brought out from the vehicle includes a sensor output obtainment section, a startup judgment section, and an automatic start section. The sensor output obtainment section is configured to obtain a sensor output which is output from a sensor. The startup judgment section is configured to judge whether or not a vehicle system controlling the vehicle has started. The automatic start section is configured to cause the sensor output obtainment section to start obtainment of the sensor output when the startup judgment section judges that the vehicle system has started.

### CROSS-REFERENCE TO RELATED APPLICATION

This application claims priority from Japanese Patent Application No. 2018-068295 filed on Mar. 30, 2018, the entire subject-matter of which is incorporated herein by reference.

### TECHNICAL FIELD

The present disclosure relates to a portable measurement apparatus which can be brought into a vehicle.

### BACKGROUND

JP-A-2017-133876 describes a measurement apparatus which is mounted on a vehicle and which includes a plurality of measurement modules connected to sensors in one-to-one relation and adapted to obtain sensor outputs output from the sensors and output the obtained sensor outputs to the outside, and a main body portion collecting data output from the plurality of measurement modules.

However, in the case where measurement is performed through use of the measurement apparatus described in JP-A-2017-133876 while the vehicle is caused to travel, a measurement operator must perform a start operation before causing the vehicle to travel. The start operation includes bringing the power switch of the measurement apparatus into an on state and then causing the measurement modules to start sensor control.

### SUMMARY

The present specification discloses a measurement apparatus which enables to improve the convenience of measurement.

One mode of the present disclosure is a portable measurement apparatus which can be brought into a vehicle and brought out from the vehicle and which includes a sensor output obtainment section, a startup judgment section, and an automatic start section.

The sensor output obtainment section is configured to obtain a sensor output which is output from a sensor. The startup judgment section is configured to judge whether or not a vehicle system controlling the vehicle has started. The automatic start section is configured to cause the sensor output obtainment section to start obtainment of the sensor output when the startup judgment section judges that the vehicle system has started.

In the measurement apparatus of the present disclosure configured as described above, when the vehicle system starts, obtainment of the sensor output is automatically started even when a measurement operator does not perform a start operation. Therefore, the measurement apparatus of the present disclosure can free the measurement operator from the requirement of performing the start operation, thereby improving the convenience of measurement.

Also, in one mode of the present disclosure, the measurement apparatus may include an automatic judgment section and a prohibition section. The automatic judgment section is configured to judge whether or not the measurement apparatus has been set to an automatic mode. The prohibition section is configured to prohibit the automatic start section from starting the obtainment of the sensor output when the automatic judgment section judges that the measurement apparatus has not been set to the automatic mode. By virtue of this, the measurement apparatus of the present disclosure can cause the measurement operator to select whether to automatically start the obtainment of the sensor output when the vehicle system starts.

Also, in one mode of the present disclosure, the startup judgment section may include a relay having a coil and a switch. In one mode of the present disclosure, the relay may be configured such that when the vehicle system is in a started state, current flows through the coil, so that the switch is brought into an on state, and when the vehicle system is in a stopped state, no current flows through the coil, so that the switch is brought into an off state. By virtue of this, the measurement apparatus of the present disclosure can simply and easily judge whether or not the vehicle system has been started by judging whether or not the switch of the relay is in the on state.

Also, in one mode of the present disclosure, the measurement apparatus may be configured such that voltage is supplied from a battery provided externally of the measurement apparatus, and the switch of the relay is disposed in a voltage supply path provided between the battery and the measurement apparatus. By virtue of this, when the vehicle system starts, voltage is supplied from the battery to the measurement apparatus of the present disclosure as a result of the switch of the relay being brought into the on state. Further, when the vehicle system stops, the supply of voltage from the battery to the measurement apparatus of the present disclosure is stopped as a result of the switch of the relay being brought into the off state. Therefore, the measurement apparatus of the present disclosure can eliminate the necessity of operation of bringing the power switch of the measurement apparatus into the on state when measurement is to be started. Furthermore, the measurement apparatus of the present disclosure can eliminate the necessity of operation of bringing the power switch of the measurement apparatus into the off state when measurement is to be ended. Therefore, the measurement apparatus of the present disclosure can free the measurement operator from the requirement of operating the power switch, thereby further improving the convenience of measurement.

### BRIEF DESCRIPTION OF DRAWINGS

FIG. 1 shows the schematic configuration of a measurement system.

FIG. 2 shows the schematic configuration of a DC relay.

FIG. 3 shows perspective view of an apparatus main body.

FIG. 4 shows perspective view of a module and a sensor.

FIG. 5 shows perspective view of a main unit.

FIG. 6 shows the configurations of the main unit and the module.

FIG. 7 shows a flowchart showing an automatic start process.

### DETAILED DESCRIPTION

An embodiment of the present disclosure will now be described with reference to the drawings.

A measurement system 1 of the present embodiment is mounted on a vehicle and includes a measurement apparatus 100 and a battery 400 as shown in FIG. 1.

The battery 400 is provided so as to supply power supply voltage to the measurement apparatus 100, separately from an on-board battery which is mounted on the vehicle beforehand. Notably, the power supply voltage may be supplied from the on-board battery to the measurement apparatus 100.

The measurement apparatus 100 includes an apparatus main body 200 and a DC relay 300.

The apparatus main body 200 operates while receiving voltage from the battery 400 through the DC relay 300.

The DC relay 300 is connected to a cigar socket CS of the vehicle. The DC relay 300 is in an on state in a state in which the DC relay 300 receives voltage from the cigar socket CS. As a result, voltage is supplied from the battery 400 to the apparatus main body 200. Meanwhile, the DC relay 300 is in an off state in a state in which the DC relay 300 does not receive voltage from the cigar socket CS. As a result, the supply of voltage from the battery 400 to the apparatus main body 200 is stopped.

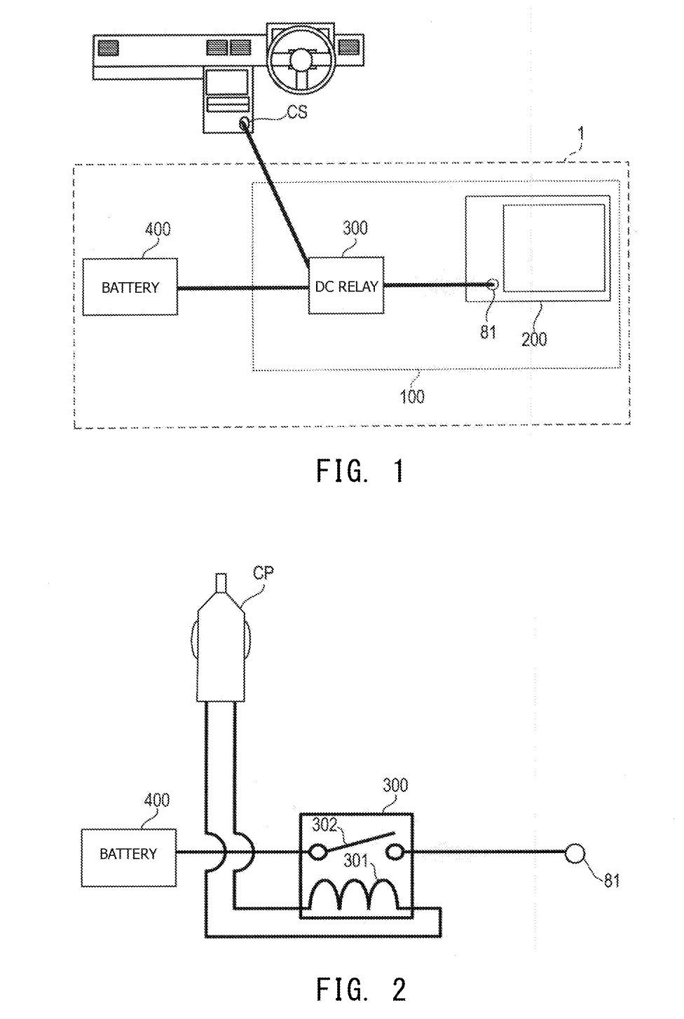

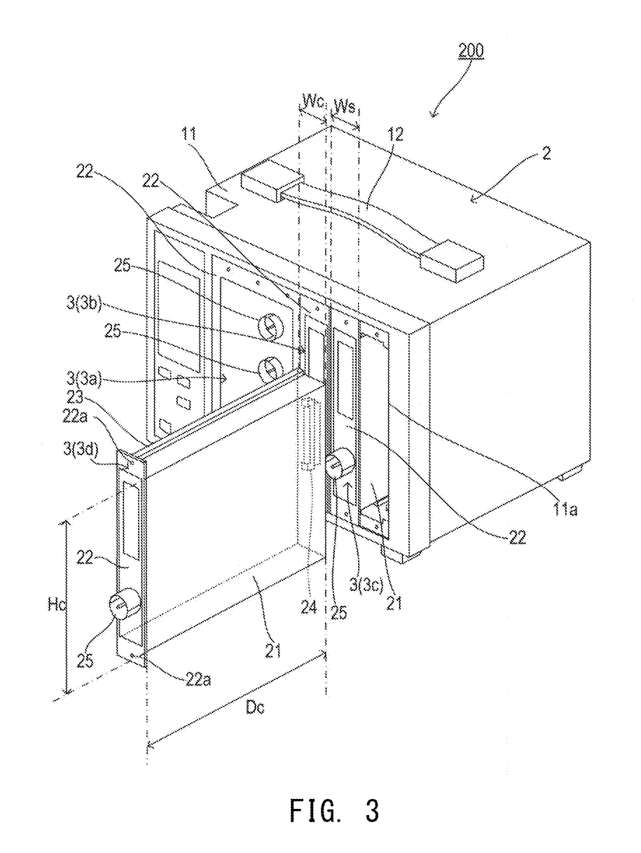

As shown in FIG. 2, the DC relay 300 includes a coil 301 and a switch 302.

One end of the coil 301 is connected to a plus-side wire of a cigar plug CP, and the other end of the coil 301 is connected to a minus-side wire of the cigar plug CP. The cigar plug CP is inserted into the cigar socket CS.

One end of the switch 302 is connected to the battery 400, and the other end of the switch 302 is connected to the apparatus main body 200.

When a driver inserts a key for the vehicle into an ignition key cylinder and turns the inserted key such that the key switch is switched to an accessory (hereinafter referred to as “ACC”) position or an ignition (hereinafter referred to as “IG”) position, the power supply voltage of the on-board battery is supplied from the cigar socket CS. As a result of switching of the key switch to the ACC position or the IG position, the power supply voltage of the on-board battery is supplied to a vehicle system which controls equipments mounted on the vehicle, so that the vehicle system starts. An example of the vehicle system is an electronic control apparatus for controlling an engine mounted on the vehicle.

When voltage is supplied to the coil 301, the switch 302 is brought into the on state, so that voltage is supplied from the battery 400 to the apparatus main body 200.

After that, when the driver rotates the inserted key so as to switch the key switch to an off position, the supply of the power supply voltage of the on-board battery through the cigar socket CS is stopped. When the supply of voltage to the coil 301 is stopped, the switch 302 is brought into the off state, so that the supply of voltage from the battery 400 to the apparatus main body 200 is stopped.

As shown in FIG. 3, the apparatus main body 200 includes one main unit 2 and four modules 3a, 3b, 3c, and 3d. In the following description, one module which represents the modules 3a. 3b, 3c, and 3d will be referred to as a “module 3.”

The main unit 2 includes a housing 11 and a handle 12.

The housing 11 has a box-like shape of rectangular parallelepiped (in the present embodiment, for example, 30 cm (height)×40 cm (width)×30 cm (depth)) and accommodates the components of the main unit 2 and the module 3 therein.

A rectangular opening 11a is formed in the front surface among the six surfaces which constitute the rectangular parallelepiped of the housing 11. The module 3 is inserted through the opening 11a, whereby the module 3 is accommodated in the housing 11.

The handle 12 is attached to the top surface among the six surfaces which constitute the rectangular parallelepiped of the housing 11. A user of the main unit 2 can carry the main unit 2 by grasping the handle 12.

The module 3a is an apparatus into which a portion of exhaust gas discharged from a diesel engine is introduced and which measures the amount of particulate matters contained in the exhaust gas of the diesel engine. The module 3b is an apparatus which measures the concentration of nitrogen oxides contained in the exhaust gas through use of an NOx sensor. The module 3c is an apparatus which measures the concentration of ammonia contained in the exhaust gas through use of an ammonia sensor. The module 3d is an apparatus which measures the air-fuel ratio of the exhaust gas through use of an air-fuel ratio sensor.

The module 3 includes a casing 21, a mounting plate 22, guide rails 23, a unit connection connector 24, and a sensor connection connector 25.

The casing 21 has a box-like shape of rectangular parallelepiped and accommodates the components of the module 3 therein.

The height Hc and depth Dc of the casing 21 are set in advance such that the modules 3a, 3b, 3c, and 3d have the same dimensions, so that the module 3a, 3b, 3c, and 3d are accommodated in the housing 11 in a state in which they are lined up in a horizontal direction.

The width Wc of the casing 21 is set such that the width Wc is approximately equal to n times a slot width Ws, which is the minimum unit of the width of the module 3, where n is an integer. Notably, the width Wc of the modules 3a is about three times the slot width. The widths Wc of the module 3b, 3c, and 3d are approximately equal to the slot width.

The mounting plate 22 is a rectangular plate member having a height approximately equal to the height of the rectangular opening 11a and a width approximately equal to the width Wc of the casing 21.

The mounting plate 22 is attached to the front surface among the six surfaces which constitute the rectangular parallelepiped of the casing 21. Notably, the mounting plate 22 is disposed such that the upper side among the four sides which constitute the rectangle of the mounting plate 22 is located above the upper side among the four sides which constitute the rectangle of the front surface of the casing 21. Further, the mounting plate 22 is disposed such that the lower side among the four sides which constitute the rectangle of the mounting plate 22 is located below the lower side among the four sides which constitute the rectangle of the front surface of the casing 21.

Through holes 22a are formed in portions of the mounting plate 22 which are not in contact with the casing 21. Screws for fixing the module 3 in a state in which it is accommodated in the main unit 2 are inserted through the through holes 22a.

The guide rails 23 are attached to the top and bottom surfaces among the six surfaces which constitute the rectangular parallelepiped of the casing 21. Notably, in FIG. 3, the guide rail 23 attached to the bottom surface is not illustrated. The guide rails 23 are provided such that they extend from the front surface toward the back surface, which surfaces constitute the rectangular parallelepiped of the casing 21, and they project from the top surface and the bottom surface, respectively.

The unit connection connector 24 is a connector for connecting the module 3 to the main unit 2 and is attached to the back surface of the casing 21. The unit connection connectors 24 of the modules 3a, 3b, 3c, and 3d have the same shape.

The sensor connection connector 25 is a connector for connecting a sensor to the module 3 and is attached to the front surface of the mounting plate 22.

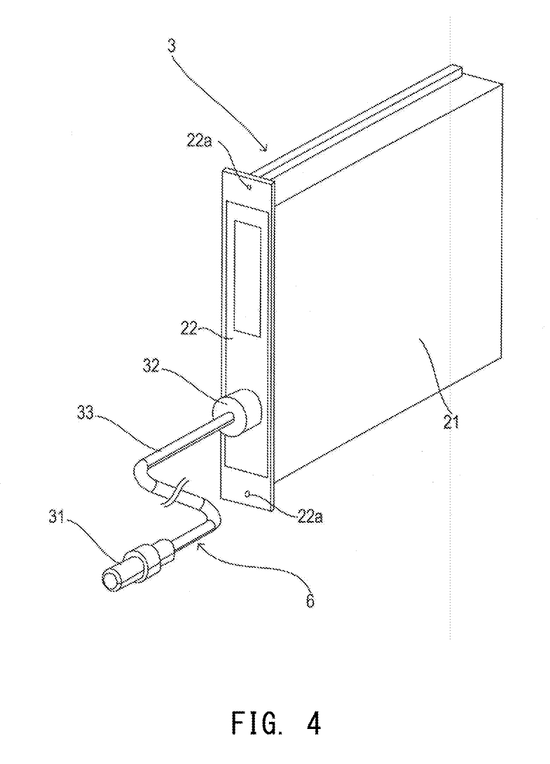

As shown in FIG. 4, a sensor 6 includes a sensor element 31, a connector 32, and a signal cable 33. The sensor element 31 detects a physical quantity corresponding to the function of the module 3 to which the sensor 6 is connected. The connector 32 has a structure which allows the connector 32 to be detachably mated with the sensor connection connector 25 of the module 3 to which the sensor 6 is connected. The signal cable 33 is a signal line for electrically connecting the sensor element 31 and the connector 32.

Therefore, as a result of the connector 32 of the sensor 6 and the sensor connection connector 25 of the module 3 being mated together, it becomes possible to input a detection signal from the sensor 6 into the module 3.

The sensors 6 connected to the modules 3b, 3c, and 3d are an NOx sensor, an ammonia sensor, and an air-fuel ratio sensor, respectively. Notably, each of the NOx sensor, the ammonia sensor, and the air-fuel ratio sensor is a direct-insertion-type sensor which is directly inserted into an exhaust pipe of an internal combustion engine.

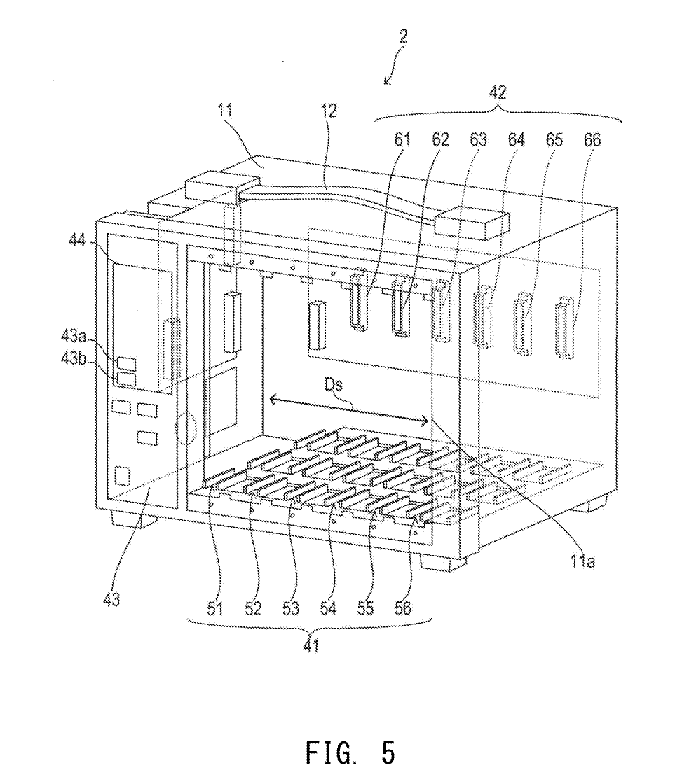

Also, as shown in FIG. 5, the main unit 2 includes a slot guide groove group 41, a module connection connector group 42, and a switch panel 43.

The slot guide groove group 41 includes slot guide grooves 51, 52, 53, 54, 55, and 56 for respective guidance to six slots set in advance.

The slot guide grooves 51 to 56 are concave portions which can be mated with the guide rails 23 provided on the top and bottom surfaces of the casing 21 of the module 3. The slot guide grooves 51 to 56 are provided such that they extend from the front surface toward the back surface, which surfaces constitute the rectangular parallelepiped of the housing 11.

Also, the slot guide grooves 51 to 56 are provided in the vicinity of each of the upper and lower sides of the rectangle of the rectangular opening 11a. Notably, in FIG. 5, the slot guide grooves 51 to 56 provided in the vicinity the upper side are not illustrated.

The slot guide grooves 51 to 56 are disposed at intervals equal to the slot width Ws along a slot arrangement direction Ds which is set in advance such that it becomes parallel to the upper and lower sides of the rectangle of the of the rectangular opening 11a.

Therefore, the module 3 can be accommodated in a slot corresponding to the slot guide grooves 51 by the following steps. First, when the module 3 located outside the housing 11 is inserted into the opening 11a, the guide rails 23 provided on the top and bottom surfaces of the casing 21 of the module 3 are fitted into the slot guide grooves 51 provided near the upper and lower sides of the opening 11a. Subsequently, in a state in which the guide rails 23 are fitted into the slot guide grooves 51, the module 3 is moved toward the interior of the housing 11 along the direction in which the slot guide grooves 51 extend. As a result, the module 3 is accommodated in the housing 11.

Notably, the modules 3 can be accommodated in slots corresponding to the slot guide grooves 52, 53, 54, 55, and 56 by the above-described steps. Hereinafter, the slots corresponding to the slot guide grooves 51, 52, 53, 54, 55, and 56 will be referred to as first, second, third, fourth, fifth, and sixth slots.

The module connection connector group 42 includes module connection connectors 61, 62, 63, 64, 65, and 66. The module connection connector 61, 62, 63, 64, 65, and 66 are connectors for connecting the modules 3 accommodated in the first, second, third, fourth, fifth, and sixth slots to the main unit 2.

The module connection connectors 61 to 66 are disposed at respective positions determined such that the module connection connectors 61 to 66 can be mated with the corresponding unit connection connectors 24 disposed on the back surfaces of the modules 3 when the modules 3 are accommodated in the first to sixth slots.

The switch panel 43 includes a plurality of switches for instructing operations of the main unit 2, and a liquid-crystal display section 44 and a plurality of LED lamps for showing the operation state of the main unit 2, etc. The switch panel 43 is disposed on the front surface among the six surfaces of the rectangular parallelepiped of the housing 11. LED is an abbreviation for Light Emitting Diode. Notably, the liquid-crystal display section 44 provides various types of displays and functions as a touch panel.

Operation buttons 43a and 43b are set on the display screen of the liquid-crystal display section 44.

The operation button 43a is operated by a measurement operator to start the measurement by the measurement apparatus 100 and to end the measurement in the case where the measurement apparatus 100 has been set to a non-automatic mode to be described later. Namely, when the operation button 43a is operated in a state in which the measurement apparatus 100 does not perform measurement, the measurement by the measurement apparatus 100 is started. Also, when the operation button 43a is operated in a state in which the measurement apparatus 100 is performing measurement, the measurement by the measurement apparatus 100 is ended.

The operation button 43b is operated when the measurement apparatus 100 is set to the automatic mode and when the measurement apparatus 100 is set to the non-automatic mode. Namely, when the operation button 43b is operated in a state in which the measurement apparatus 100 has been set to the automatic mode, the measurement apparatus 100 is set to the non-automatic mode, and when the operation button 43b is operated in a state in which the measurement apparatus 100 has been set to the non-automatic mode, the measurement apparatus 100 is set to the automatic mode.

The automatic mode is a mode in which, when voltage is supplied to the measurement apparatus 100 and the measurement apparatus 100 starts, the measurement apparatus 100 automatically starts measurement even when the operation button 43a is not operated. The non-automatic mode is a mode in which the measurement apparatus 100 starts measurement when the operation button 43a is operated after the measurement apparatus 100 has started upon supply of voltage to the measurement apparatus 100.

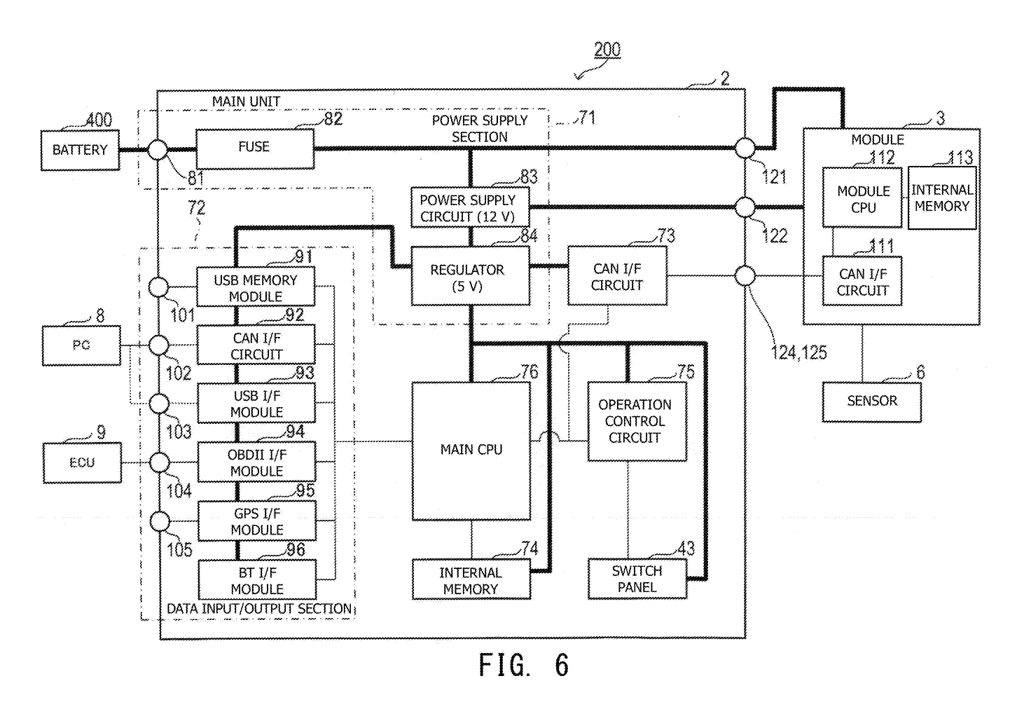

Also, as shown in FIG. 6, the main unit 2 includes a power supply section 71, a data input/output section 72, a CAN interface circuit (hereinafter referred to as the CAN I/F circuit) 73, an internal memory 74, an operation control circuit 75, and a main CPU 76. CAN is an abbreviation for Controller Area Network. CPU is an abbreviation for Central Processing Unit. CAN is a registered trademark in Japan.

The power supply section 71 includes a power supply connector 81, a fuse 82, a power supply circuit 83, and a regulator 84.

The power supply connector 81 is a connector which is connected to the battery 400 so as to receive the power supply voltage from the battery 400.

The fuse 82 is provided in a power supply path between the power supply connector 81 and VB terminals 121 of the module connection connectors 61 to 66. When an excessively large current flows through the power supply path, the fuse 82 is blown out.

The power supply circuit 83 receives the power supply voltage from the battery 400 through the fuse 82, generates a voltage of 12 V from the power supply voltage, and outputs the generated voltage (12 V) from 12-V terminals 122 of the module connection connectors 61 to 66.

The regulator 84 receives the 12 V voltage from the power supply circuit 83 and generates a voltage of 5 V. The regulator 84 outputs the generated voltage (5 V) to the data input/output section 72, the CAN I/F circuit 73, the internal memory 74, the operation control circuit 75, the main CPU 76, and the switch panel 43.

The data input/output section 72 includes a USB memory module 91, a CAN I/F circuit 92, a USB interface module 93, an OBD2 interface module 94, a GPS interface module 95, and a Bluetooth interface module 96. USB is an abbreviation for Universal Serial Bus. OBD is an abbreviation for On Board Diagnosis. GPS is an abbreviation for Global Positioning System. Bluetooth is a registered trademark.

Hereinafter, the USB interface module 93, the OBD2 interface module 94, the GPS interface module 95, and the Bluetooth interface module 96 will be referred to as the USB I/F module 93, the OBD2 I/F module 94, the GPS I/F module 95, and the BT I/F module 96, respectively.

Also, the data input/output section 72 includes a USB memory connector 101, a CAN communication connector 102, a USB connector 103, an OBD2 connector 104, and a GPS connector 105.

In accordance with a scheme conforming to the USB standard, the USB memory module 91 sends data to and receives data from a USB memory connected through the USB memory connector 101.

In accordance with the CAN communication protocol, the CAN I/F circuit 92 sends data to and receives data from a device (for example, a personal computer 8) connected through the CAN communication connector 102.

In accordance with a scheme conforming to the USB standard, the USB I/F module 93 sends data to and receives data from a device connected through the USB connector 103.

In accordance with a scheme conforming to the OBD2 standard, the OBD2 I/F module 94 sends data to and receives data from a device (for example, an onboard ECU 9) connected through the OBD2 connector 104. ECU is an abbreviation for Electronic Control Unit.

The GPS I/F module 95 is an interface which allows a GPS receiver for receiving satellite signals from GPS satellites to be connected to the main unit 2 through the GPS connector 105. Notably, in FIG. 6, the GPS receiver is not illustrated.

The BT I/F module 96 performs short distance wireless communications in accordance with a scheme conforming to the Bluetooth standard.

In accordance with the CAN communication protocol, the CAN I/F circuit 73 sends data to and receives data from the modules 3 connected to CAN_H terminals 124 and CAN_L terminals 125 of the module connection connectors 61 to 66.

The internal memory 74 is a storage device for storing various data. The internal memory 74 includes a ROM and a RAM.

The operation control circuit 75 outputs to the main CPU 76 input operation information for specifying input operations performed by a user through the switches of the switch panel 43 and the touch panel of the liquid-crystal display section 44. Also, the operation control circuit 75 controls the operations of the liquid-crystal display section 44 and the LED lamps of the switch panel 43 on the basis of instructions from the main CPU 76.

The main CPU 76 executes various types of processing on the basis of inputs from the data input/output section 72, the CAN I/F circuit 73, the internal memory 74, and the operation control circuit 75 and controls the data input/output section 72, the CAN I/F circuit 73, the internal memory 74, and the operation control circuit 75.

The various types of functions of the main unit 2 are realized by a program which is stored in a non-transitory tangible recording medium and executed by the main CPU 76. In this example, the ROM of the internal memory 74 corresponds to the non-transitory tangible recording medium storing the program. Also, a method corresponding to the program is performed as a result of execution of this program. Notably, some or all of the functions of the main CPU 76 may be realized by hardware; for example, by a single IC or a plurality of ICs.

For example, the main CPU 76 stores in the internal memory 74 the measurement data received from the modules 3 through the CAN I/F circuit 73.

Also, the main CPU 76 stores the measurement data received from the modules 3 in a USB memory connected to the USB memory module 91.

Also, the main CPU 76 outputs the measurement data received from the modules 3 to the personal computer 8 connected to the CAN I/F circuit 92 or the USB I/F module 93.

Also, the main CPU 76 stores in the internal memory 74 the data received from the on-board ECU 9 connected to the OBD2 I/F module 94.

Also, the main CPU 76 calculates the present position of the main unit 2 on the basis of the satellite signals received from the GPS receiver connected to the GPS connector 105, and stores the calculated present position in the internal memory 74.

Also, the main CPU 76 uses the BT I/F module 96 so as to send the measurement data received from the modules 3 by means of short distance wireless communication.

Also, when the main CPU 76 receives measurement setting information, which shows the measurement conditions under which each module 3 performs measurement, from the personal computer 8 connected to the CAN I/F circuit 92 or the USB I/F module 93, the main CPU 76 sends the measurement setting information to the module 3 through the CAN I/F circuit 73. As a result, in the module 3 having received the measurement setting information, the measurement conditions are changed such that the module 3 performs measurement under the measurement conditions indicated by the measurement setting information.

Next, the module 3 includes a CAN I/F circuit 111, a module CPU 112, and an internal memory 113.

The CAN I/F circuit 111 sends data to and receives data from the main unit 2 in accordance with the CAN communication protocol.

The module CPU 112 executes various types of processing on the basis of inputs from the sensor 6 and the CAN I/F circuit 111 and controls the sensor 6 and the CAN I/F circuit 111.

The internal memory 113 is a storage device for storing various data.

In the apparatus main body 200 configured as described above, the main CPU 76 executes an automatic start process.

Next, the steps of the automatic start process executed by the main CPU 76 will be described. The automatic start process is started immediately after the main CPU 76 has started upon supply of voltage from the battery 400 through the DC relay 300.

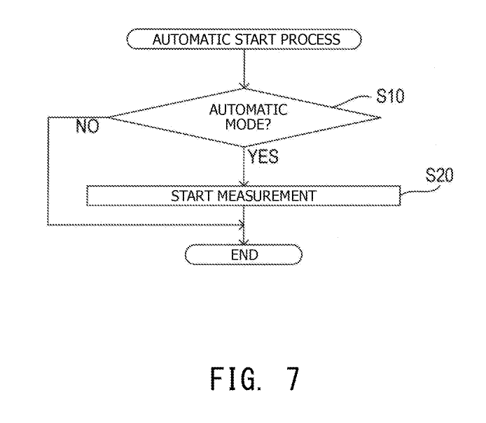

When the automatic start process is executed, as shown in FIG. 7, the main CPU 76 first judges in S10 whether or not the measurement apparatus 100 has been set to the automatic mode. In the case where the measurement apparatus 100 has been set to the automatic mode, in S20, the main CPU 76 starts the measurement by the apparatus main body 200. Specifically, the main CPU 76 sends an instruction for instructing the start of measurement to the modules 3 connected to the apparatus main body 200. As a result, the modules 3 start measurement. Also, the main CPU 76 starts a process of receiving measurement data from the modules 3 by using the CAN I/F circuit 73. When the processing of S20 ends, the main CPU 76 ends the automatic start process.

The measurement apparatus 100 configured as described above is a portable measurement apparatus which can be brought into the vehicle or brought out from the vehicle.

The apparatus main body 200 of the measurement apparatus 100 obtains measurement data output from the modules 3. The DC relay 300 of the measurement apparatus 100 judges whether or not the vehicle system for controlling the vehicle has started. In the case where the vehicle system is judged to have started, the apparatus main body 200 of the measurement apparatus 100 starts the obtainment of the measurement data.

As described above, in the measurement apparatus 100, when the vehicle system starts, the obtainment of the measurement data is automatically started even when the measurement operator does not perform the start operation. Therefore, the measurement apparatus 100 can free the measurement operator from the requirement of performing the start operation, thereby improving the convenience of measurement.

Also, the apparatus main body 200 of the measurement apparatus 100 judges whether or not the measurement apparatus 100 has been set to the automatic mode. In the case where the apparatus main body 200 judges that the measurement apparatus 100 has not been set to the automatic mode, the apparatus main body 200 prohibits the automatic starting of the obtainment of the measurement data. As a result, the measurement apparatus 100 can cause the measurement operator to select whether to automatically start the obtainment of the measurement data when the vehicle system starts.

Also, the DC relay 300 includes the coil 301 and the switch 302. The DC relay 300 is configured such that when the vehicle system is in its started state, current flows through the coil 301, whereby the switch 302 is brought into the on state, and when the vehicle system is in its stopped state, no current flows through the coil 301, whereby the switch 302 is brought into the off state. Therefore, the measurement apparatus 100 can simply and easily judge whether or not the vehicle system has been started by judging whether or not the switch 302 of the DC relay 300 is in the on state.

Also, the measurement apparatus 100 is configured such that voltage is supplied from the battery 400 provided externally of the measurement apparatus 100, and the switch 302 of the DC relay 300 is disposed in the voltage supply path provided between the battery 400 and the apparatus main body 200. As a result, when the vehicle system starts, voltage is supplied from the battery 400 to the measurement apparatus 100 as a result of the switch 302 of the DC relay 300 being brought into the on state. Further, when the vehicle system stops, the supply of voltage from the battery 400 to the measurement apparatus 100 is stopped as a result of the switch 302 of the DC relay 300 being brought into the off state. As a result, the measurement apparatus 100 can eliminate the necessity of operation of bringing the power switch of the apparatus main body 200 into the on state when measurement is to be started. Further, the measurement apparatus 100 can eliminate the necessity of operation of bringing the power switch of the apparatus main body 200 into the off state when measurement is to be ended. Therefore, the measurement apparatus 100 can free the measurement operator from the requirement of operating the power switch, thereby further improving the convenience of measurement.

In the above-described embodiment, the CAN I/F circuit 73 corresponds to the sensor output obtainment section; the DC relay 300 corresponds to the startup judgment section; S20 corresponds to the processing as the automatic start section, the modules 3 correspond to the sensor, and the measurement data corresponds to the sensor output.

Also, S10 corresponds to the processing as the automatic judgment section and the prohibition section; and the DC relay 300 corresponds to the relay.

One embodiment of the present disclosure has been described; however, the present disclosure is not limited to the above-described embodiment and various modifications are possible.

For example, in the above-described embodiment, the vehicle system starts as a result of the key switch being switched to the ACC position or the IG position. However, the present disclosure may be applied to a vehicle whose vehicle system starts when a driver carrying a smart key operates a start switch disposed inside the vehicle.

Also, in the above-described embodiment, the judgment as to whether or not the vehicle system has been started is made by judging whether or not the switch 302 of the DC relay 300 is in the on state. However, the above-described embodiment may be configured such that the apparatus main body 200 is connected to an output terminal whose voltage changes from a low level to a high level when the key switch is switched to the ACC position or the IG position, and the judgment as to whether or not the vehicle system has been started is made by judging whether or not the voltage of the output terminal is the high level. Alternatively, the above-described embodiment may be configured such that the apparatus main body 200 is connected to the OBD2 terminal, and the judgment as to whether or not the vehicle system has been started is made on the basis of a vehicle signal output from the OBD2 terminal.

Also, in the above-described embodiment, the start of measurement and the setting of the automatic mode or the non-automatic mode are instructed by operating the operation buttons 43a and 43b set on the display screen of the liquid-crystal display section 44. However, mechanical push buttons may be used in place of the operation buttons set on the display screen.

Also, the function of one constituent element in the above-described embodiment may be distributed to a plurality of constituent elements, or the functions of a plurality of constituent elements may be realized by one constituent element. Part of the configuration of the above-described embodiment may be omitted. Also, at least part of the configuration of the above-described embodiment may be added to or partially replace the configurations of other embodiments. Notably, all modes included in the technical idea specified by the wording of the claims are embodiments of the present disclosure.

The present disclosure may be realized in various forms other than the above-described measurement apparatus 100. For example, the present disclosure may be realized as a system including the measurement apparatus 100 as a constituent element, a program for causing a computer to function as the measurement apparatus 100, a medium on which the program is recorded, and a measurement method.

What is claimed is:

1. A portable measurement apparatus which can be brought into a vehicle and brought out from the vehicle, comprising:
a sensor output obtainment section configured to obtain a sensor output which is output from a sensor;
a startup judgment section configured to judge whether or not a vehicle system controlling the vehicle has started; and
an automatic start section configured to cause the sensor output obtainment section to start obtainment of the sensor output when the startup judgment section judges that the vehicle system has started.

2. The measurement apparatus according to claim 1, further comprising:
an automatic judgment section configured to judge whether or not the measurement apparatus has been set to an automatic mode; and
a prohibition section configured to prohibit the automatic start section from starting the obtainment of the sensor output when the automatic judgment section judges that the measurement apparatus has not been set to the automatic mode.

3. The measurement apparatus according to claim 1, wherein the startup judgment section includes a relay having a coil and a switch, and the relay is configured such that when the vehicle system is in a started state, current flows through the coil, so that the switch is brought into an on state, and when the vehicle system is in a stopped state, no current flows through the coil, so that the switch is brought into an off state.

4. The measurement apparatus according to claim 3, wherein voltage is supplied from a battery provided externally of the measurement apparatus, and the switch of the relay is disposed in a voltage supply path provided between the battery and the measurement apparatus.
